(19) United States
(12) Patent Application Publication
(10) Pub. No.: US 2024/0165209 A1
(43) Pub. Date: May 23, 2024

(54) PLACENTAL ALKALINE PHOSPHATASE FOR REDUCING THE LOSS OF RED BLOOD CELLS, WHITE BLOOD CELLS, AND PLATELETS IN CHEMOTHERAPY TREATED CANCER PATIENTS

(71) Applicant: ZOLTAN LABORATORIES, LLC, Albert Lea, MN (US)

(72) Inventor: Zoltan Kiss, Austin, MN (US)

(21) Appl. No.: 18/277,214

(22) PCT Filed: Feb. 16, 2021
(86) PCT No.: PCT/US2021/018164

§ 371 Date: Aug. 14, 2023

(51) Int. Cl. A61K 38/46; A61P 7/00

(52) U.S. Cl. CPC A61K 38/465; A61P 7/00; C12Y 301/03001

(57) ABSTRACT

This disclosure provides recombinant placental alkaline phosphatase and highly purified human placental alkaline phosphatase for the treatment of a chemotherapy treated cancer patient to reduce chemotherapy-induced loss of red blood cells, white blood cells, or platelets as well as increase the survival of chemotherapy challenged hematopoietic stem cells, mesenchymal stem cells, or blood cell forming progenitors. Another use of recombinant placental alkaline phosphatase or placental alkaline phosphatase for chemotherapy treated cancer patients is to enhance survival of transplanted hematopoietic and mesenchymal stem cells thereby promoting formation of blood cells. The novelty of this disclosure is using placental alkaline phosphatases to reduce the loss of these cells despite the presence of a highly proteolytic environment in the bone marrow created by chemotherapy.

### BACKGROUND

This disclosure provides recombinant placental alkaline phosphatase and highly purified human placental alkaline phosphatase to reduce the loss of red blood cells, white blood cells, and platelets in chemotherapy treated cancer patients. Placental alkaline phosphatases reduces the loss of these cells despite the highly proteolytic environment in the bone marrow created by chemotherapy.

### Summary of Major Forms of Chemotherapy Induced Cytopenia.

Generally, chemotherapy damages bone marrow cells resulting in various degrees of cytopenia, defined as deficiency in the number of any of the cellular elements of the blood. Chemotherapy also causes multitude of effects on the structural elements of the bone marrow. In addition, chemotherapy creates a highly proteolytic microenvironment in the bone marrow which goes along with a decrease of endogenous protease inhibitors [Maurer, A., Klein, G. and Staudt, N. D. Assessment of proteolytic activities in the bone marrow microenvironment. Methods Mol. Biol. 2017, 149-163, 2019]. These two processes may reduce the concentration of bioactive proteins in the bone marrow. The major forms of cytopenia are anemia, neutropenia, febrile neutropenia, and thrombocytopenia.

Anemia is a condition when the blood does not contain enough red blood cells (erythrocytes) and, thus, inadequate amount of oxygen is carried to the body's tissues. The person with anemia feels tired and weak. Chemotherapeutic drugs can cause anemia by reducing formation or viability of red blood cells. Some types of anemia are not drug related, and these are out of the scope of this disclosure. Long-lasting anemia can damage the heart, brain, and other organs, while severe anemia may even cause death. Importantly, low red blood cell counts not only lead to fatigue and reduced tolerance to cancer therapy, but it also promotes hypoxia which is an established stimulus for tumor growth [see, for example, Zhong, H., De Marzo, A. M., Laughner, E., Lim, M., Hilton, D. A., Zagzag, D., Buehler, P., Isaacs, W. B., Semenza, G. I. and Simons, J. W. (1999) Overexpression of hypoxia-inducible factor 1α in common human cancers and their metastases. Cancer Res. 59, 5830-5835]. A drug enabled reduction of chemotherapy induced loss of red blood cells can be lifesaving.

Aplastic anemia is the consequence of drastic decrease in the production of all types of blood cells. A person with this disease may be a candidate for bone marrow transplantation.

Leukopenia is an umbrella term that refers to a reduction in any of the white blood cell types (neutrophils, monocytes, basophils, eosinophils, lymphocytes, macrophages).

Granulocytopenia (granulomatous disease) is an acute condition involving a severe and dangerous leukopenia, leading to reduced numbers of neutrophils, monocytes, and macrophages in the circulating blood. Granulocytopenia, frequently caused by chemotherapy such as alkylating agents like cyclophosphamide (Cyp), decreases the body's defense against bacterial or fungal infection [Ballestrero, A., Ferrando, F., Garuti, A., Basta, P., Gonella, R., Stura, P., Mela, G. S., Sessarego, M, Gobbi, M. and Patrone, F. Comparative effects of three cytokine regimens after high-dose cyclophosphamide: Granulocyte colony-stimulating factor, granulocyte-macrophage colony-stimulating factor (GM-CSF), and sequential interleukin-3 and GM-CSF. J. Clin. Oncol. 17, 1296-1303, 1999; The International Collaborative Ovarian Neoplasm (ICON) Group (2002); Paclitaxel plus carboplatin versus standard chemotherapy with either single-agent carboplatin or cyclophosphamide, doxorubicin, and cisplatin in women with ovarian cancer: the ICON3 randomised trial. Lancet 360, 505-515].

In agranulocytosis, the number of granulocytes (neutrophils, basophils, and eosinophils) drops below 500 cells/mm³ of blood. In this condition, rapid restoration of bone marrow function is particularly important because of extreme vulnerability to infection which can lead to death.

Neutropenia, another subcategory of leukopenia, is a condition when the concentration of neutrophils in the blood is abnormally low. Neutrophils make up 60-70 percent of the circulating white blood cells and serve as the primary defense against bacterial and viral infections.

Febrile neutropenia is a form of neutropenia when the patient develops fever; bacteremia (bacteria in the bloodstream) occurs in 20% of patients with this condition which may lead to sepsis. Again, a drug that can reduce leukopenia, and particularly neutropenia and febrile neutropenia is expected to reduce dangerous infections and contribute to increased life expectancy of subjects with cancer.

Thrombocytopenia: This disease is caused by a shortage of platelets that often results in severe, life threating, bleeding of internal or external organs. It has two types, namely idiopathic and secondary ones. In idiopathic thrombocytopenia, the spleen and lymph tissues produce antibodies that destroy the platelets prematurely. Secondary thrombocytopenia may be caused, among others, by many chemotherapeutic agents. It is important to use a drug to counter thrombocytopenia and avoid a severe life-threatening condition.

Lymphocytopenia: In this condition, the number of lymphocytes (T lymphocytes, B lymphocytes, natural killer cells) in the blood is reduced with one of the major consequences that the immune system provides less protection against bacteria, viruses, fungi, and parasites. Bone marrow damaging chemotherapy, such as performed, for example, with alkylating drugs, can lead to lymphocytopenia which may result in death.

Hematopoietic progenitors give rise to the formation of homogenous populations of blood cells. Thus, granulocyte-colony forming unit (CFU-G) cells give rise to a homogenous population of neutrophils, basophils and eosinophils. M-colony forming unit (CFU-M) cells give rise to a homogeneous population of macrophages, while granulocyte, macrophage-colony forming unit (CFU-GM, often named as GM-CFU cells), give rise to macrophages and various subclasses of granulocytes. The viability of all these progenitors can be seriously reduced by chemotherapy.

### Dependence of Hematopoiesis on Hematopoietic Stem Cells (HSC) and the Bone Marrow Niche.

Hematopoietic stem cells (HSCs) possess multipotentiality, enabling them to both self-renew in the bone marrow and produce mature blood cells, such as red blood cells, white blood cells or leukocytes (neutrophils, monocytes, basophils, eosinophils, lymphocytes, macrophages) and platelets. CD34 is a marker of human HSCs and all colony-forming activity of human bone marrow (BM) cells. In this-disclosure, “BM” cells mean nucleated bone marrow cells mostly composed of HSCs and some other minor fractions including mesenchymal stem cells (MSCs) and colony forming cells.

The multi-potent MSCs are also important components of the bone marrow niche supporting hematopoiesis via releasing various molecules that play crucial roles in proliferation, differentiation, homing, migration, and self-renewal of hematopoietic stem cells [Agmasheh, S., Shamsasanjan, K., Akbarzadehlaleh, P., Sarvar, D. P. and Timari, H. Effects of mesenchymal stem cell derivatives on hematopoiesis and hematopoietic stem cells. Adv. Pharm. Bull. 7, 165-177, 2017; Spees, J. L., Lee, R. H. and Gregory, C. A. Mechanisms of mesenchymal stem/stromal cell function. Stem Cell Research & Therapy. 7, 125, DOI 10.1186/s 13287-016-0363-7, 2016].

Relevant to this disclosure is that chemotherapy results in the release of ATP from damaged and dying cells and that extracellular ATP reduces survival of MSCs [Coppi, E., Pugliese, A. M., Urbani, S. et al. ATP modulates cell proliferation and elicits two different electrophysiological responses in human mesenchymal stem cells. Stem Cells. 25, 1840-1849, 2007]. Thus, neutralization of inhibitory effects of ATP on the survival of bone marrow's MSCs is expected to increase hematopoiesis in chemotherapy treated cancer patients.

MSCs secrete numerous factors that affect the proliferation of other cell types as well as the immune system. One such factor, insulin like growth factor-1 (IGF-1) also enhances proliferation of MSCs [Huat, T. J., Khan, A. A., Pati, S. et al. IGF-1 enhances cell proliferation and survival during early differentiation of mesenchymal stem cells to neural progenitor-like cells. BMC Neuroscience. 15:91, http://www.biomedcentral.com/1471-2202/15/91, 2014].

It is noteworthy and relevant to this disclosure that compared to HSCs, MSCs are less sensitive to certain chemotherapy drugs such as cyclophosphamide (Cyp) [Li, J., Law, H. K. W., Lau, Y. L. and Chan, G. C. F. Differential damage and recovery of human mesenchymal stem cells after exposure to chemotherapeutic agents. J. Hematology. 127, 326-334, 2004]. This means that after chemotherapy treatment relatively more MSCs will remain functional compared to HSCs, particularly if a drug is also used to neutralize the inhibitory effect of extracellular ATP and chemotherapy on the proliferation of MSCs. Such drug would be expected to further facilitate regeneration of bone marrow when HSCs are also present in critical numbers.

### Myelotoxicity Related to the Use of Chemotherapy.

The number of nucleated bone marrow cells (BM) and granulocyte-macrophage colony-forming Units (CFU-GM) is a good indication of the health of the bone marrow. Chemotherapy tends to decrease, while protective agents tend to increase the number of BM and CFU-GM in the bone marrow. Proliferating HSCs and progenitor cells are responsible for the repopulation of bone marrow after chemotherapy. If a protective drug reduces the loss and enhances recovery of blood cells between cycles of chemotherapy, this means that the length of time between chemotherapy sessions can be decreased allowing the more frequent use of chemotherapy that will increase the chances of patient's survival.

Many established anticancer drugs can cause myelotoxicity. Some examples (the following does not represent the full list) for such prominent cancer drugs that affect DNA synthesis and cause myelotoxicity of various degrees include cyclophosphamide (Cyp), cisplatin (CisPt), doxorubicin, epirubicin, altretamine, azacitidine, bleomycin, busulfan, capecitabine, carboplatin, oxoplatin, carmustine, chlorambucil, cladribine, acetyldidinaline, melphalan, clofarabine, cytarabine, dacarbazine, dactinomycin, daunorubicin, decitabine, etoposide, fluoroacyl, fludarabine, hydroxyurea, idarubicin, ifosfamide, irinotecan, mechlorethamine, methotrexate, melphalan, mitomycin, mercaptopurin, mitoxantrone, nelarabine, paclitaxel, docetaxel, lomustin, gemcitabine, gemtuzumab ozogamicin, mercaptopurine, pentostatin, procarbazine, raltitrexed, streptozocin (also named streptozotocin), teniposide, thiotepa, topotecan, and valrubicin.

In most cases, the above drugs are used in various combinations. This disclosure uses Cyp as the model chemotherapeutic agent; however, the disclosure also covers all chemotherapeutic drugs and their combinations even if they do not include Cyp, if they cause loss of red blood cells, white blood cells, and platelets.

Cyp as the Model Chemotherapeutic Drug that Reduces the Number of Blood Cells.

In this disclosure, Cyp, marketed in many countries as Cytoxan or Neosar, is used as the model anticancer compound to determine its effects on blood cells in a tumor model. Its chemical name is 2-[Bis (2-chloroethyl) amino] tetrahydro-2H-1, 3, 2-oxazaphosphorine 2-oxide monohydrate; its molecular formula is C₇H₁₅Cl₂N₂O₂P and its molecular weight is 261.1.

Cyp has been in use for more than 40 years to treat (usually in combination with other drugs) breast cancer, testicular, endometrial, ovarian, hormone dependent prostate, and lung cancers, neuroblastoma, retinoblastoma, rhabdomyosarcoma and Ewing's sarcoma, Hodgkin's lymphoma, non-Hodgkin's lymphoma, Burkitt's lymphoma, chronic lymphocytic leukemia, chronic myelocytic leukemia, acute myelocytic leukemia, acute lymphocytic leukemia, t-cell lymphoma, and multiple myeloma. Cyp is administered by intravenous injection or orally.

High dose Cyp may also be used to destroy cancer cells in the bone marrow (unfortunately along with healthy cells) followed by stem cell therapy for treating relapsed or refractory diffuse large B-cell lymphoma, acute lymphoblastic leukemia, localized B-lineage lymphoblastic lymphoma, other hematologic cancers, and any other cancer needing bone marrow replacement.

Strong myelotoxicity of high-dose Cyp is exploited as part of a myeloablative conditioning regimen prior to hematopoietic stem cell transplant [Salinger, D. H., McCune, J. S A., Ren, A. G., et al. Real-time dose adjustment of cyclophosphamide in a preparation regimen for hematopoietic cell transplant: A Bayesian pharmacokinetic approach. Clin. Cancer Res. 12, 4888-4898, 2006].

Since Cyp is particularly effective in the treatment of breast cancer, in this disclosure an animal model of human breast cancer is used to determine (a) its detrimental effects on red blood cells, white blood cell, platelets, stem cells, BM cells and CFU-GM, and (b) if a human protein, placental alkaline phosphatase (PLAP), can reduce the loss of these cells. Based on the facts that Cyp is effective in inhibiting many human tumors, and that the mechanism of blood cell generation is practically the same in all humans, it is reasonable to assume that the protection of blood cells by PLAP, shown in the Examples, can be extrapolated to other cancers as well. Its is also reasonable to assume that the protective effects of PLAP against the harmful effect of Cyp on blood cells can also be extended to other chemotherapeutic agents for which examples are provided under “Myelotoxicity related to the use of chemotherapy”.

Available Major Treatments to Reduce the Detrimental Effects of Chemotherapy on Blood Cells and their Progenitors.

Erythropoietin is effective in reducing anemia, but it does not significantly affect the numbers of platelets or blood cells of myeloid origin, so its effect is highly restrictive. In addition, in several studies, EPO was found to decrease survival of patients with breast cancer [Leyland-Jones, B., Semiglazov, V., Pawlicki, M., et al. Maintaining normal hemoglobin levels with epoetin alfa in mainly nonanemic patients with metastatic breast cancer receiving first-line hemotherapy: A survival study. J. Clin. Oncol. 23, 5960-5972, 2005], head and neck cancer [Henke, M., Mattern, D., Pepe, M., Bezay, Weissenberger, C., Werner, M. and Pajonk, F. Do erythropoietin receptors on cancer cells explain unexpected clinical findings? J. Clin. Onol. 24, 4708-4713, 2006], and lung cancer [Wright, J. R., Ung, Y. C., Pritchard, K. I., et al. Randomized, double-blind, placebo-controlled trial of erythropoietin in non-small-cell lung cancer with disease-related anemia. J. Clin. Oncol. 25, 1027-1032, 2007]. The possible reason for decreased survival, as discussed in these articles, is that at higher doses erythropoietin and other erythropoiesis-stimulating agents (such as darbepoietin and epotin alfa) appear to enhance tumor growth and increase thromboembolic (deep-vein thrombosis) risks. These observations led, in 2008, the US Food and Drug Administration's oncologic drugs advisory committee to recommended limits on the use of synthetic erythropoietin products, used to treat anemia in cancer patients.

Granulocyte-colony stimulating factor (G-CSF) or granulocyte-macrophage colony-stimulating factor (GM-CSF) can reduce the duration and severity of Cyp-induced neutropenia [Ballestrero, A., Ferrando, F., Garuti, A., et al. Comparative effects of three cytokine regimens after high-dose cyclophosphamide: Granulocyte colony-stimulating factor, granulocyte-macrophage colony-stimulating factor (GM-CSF), and sequential interleukin-3 and GM-CSF. J. Clin. Oncol. 17, 1296-1303, 1999]. Accordingly, these factors were approved by FDA in 1991 and are increasingly used for supportive care. However, some observations indicate that these growth factors enhance the incidence of acute myeloid leukemia or myelodysplastic syndrome [Hershman, D., Neugut, A. I., Jaobson, J. S., Wang, J., et al. Acute Myeloid leukemia or myelodysplastic syndrome following use of granulocyte colony-stimulating factors during breast cancer adjuvant chemotherapy. J. Natl. Cancer Inst. 99, 196-205, 2007], enhance chemotherapy-induced damage of bone marrow stem cells [Van Os, R., Robinson, S., Sheridan, T., et al. Granulocyte colony-stimulating factor enhances bone marrow stem cell damage caused by repeated administration of cytotoxic agents. Blood 92, 1950-1956, 1998], and mediate cancer pain [Scheizerhof, M., Stosser, S., Kurejova, M., et al. Hematopoietic colony-stimulating factors mediate tumor-nerve interactions and bone cancer pain. Nature Med. 15, 802-807, 2009]. Replacement of GM-CSF with a similarly effective agent with less side effects would be desirable.

Thrombopoietin, initially described as a regulator of megakaryocyte and platelet formation, is a growth factor that accelerates the recovery of all hematopoietic lineages following myelosuppressive therapies and that can expand hematopoietic stem cells after transplantation [Fox, N., Priestley, G., Papayannopoulou, T. and Kaushansky, K. Thrombopoietin expands hematopoietic stem cells after transplantation. J. Clin. Invest. 110, 389-394, 2002]. However, as discussed in [Hassan, M. N. and Waller, E. K. Treating chemotherapy-induced thrombocytopenia: Is it time for oncologists to use thrombopoietin agonists? Oncology (Williston Park) 29, 295-296, 2015], newer thrombopoietin receptor agonists cause bone marrow fibrosis and may promote growth of surviving cancer cells. Because of these concerns, current clinically approved thrombopoietin mimetics (romiplostim and eltrombopag) so far have used only in immune thrombocytopenia purpura patients but not yet in chemotherapy treated cancer patients. In the latter case, the present treatment regimens include platelet infusion and brief corticosteroid treatments.

Amifostine is yet another drug used for the protection of normal tissues. This drug, administered via the intravenous route, was shown to partially protect bone marrow and kidney in Cyp-treated or cisplatin (CisPt)-treated cancer patients [Kemp, G., Rose, P., Lurain, J., et al. (1996) Amifostine pretreatment for protection against cylophosphamide-induced and cisplatin-induced toxicities: Results of a randomized control trial in patients with advanced cancer. J. Clin. Oncol. 14, 2101-2112, 1996]. However, amifostine treatment is associated with reversible clinical hypotension as well as protracted nausea and vomiting in about 15-20% of patients which symptoms put increased alert and workload on the medical staff. It is also important that protection of bone marrow cells by amifostine is not associated with increased tissue regeneration, which probably explains why its use does not result in increased patient survival.

The above review clearly indicates that there is still a need to develop a drug that can simultaneously and safely reduce loss of red blood cells, white blood cells, and platelets thus preventing anemia, leukopenia/neutropenia and thrombocytopenia, respectively, in cancer patients treated with chemotherapy.

### Definitions

In this disclosure, the term “PLAP” refers to highly purified native placental alkaline phosphatase derived from human placenta via conventional purification methods. The term “rPLAP” refers to recombinant forms of PLAP with alkaline phosphatase catalytic activity that may contain unmodified or modified sequences as far as they reproduce the effects of PLAP described in this disclosure. In the specific rPLAP protein used in this disclosure and that can be substituted for PLAP, the 27-amino acid long glycosylphosphatidylinositol (GPI) anchor (present in purified native PLAP) is replaced with an 8-amino acid long FLAG sequence at the carboxyl terminal. Since in the disclosure this rPLAP reproduces the biological effects of PLAP, it is reasonable to assume that other active recombinant forms of PLAP with altered sequences can also be prepared. For example, other active derivatives of PLAP may also be recombinant hybrid alkaline phosphatases containing up to 50% portion of PLAP to provide more stability in the circulation, and up to 50% of another alkaline phosphatase to provide alkaline phosphatase activity.

For simplicity, the term “PLAP” and “rPLAP” also refers to other human alkaline phosphatases (APs) and their recombinant forms such as human intestinal alkaline phosphatase (IALP), human tissue-nonspecific alkaline phosphatase (TNAP), human germ cell (GCAPy) and bovine intestinal alkaline phosphatase protein as far as they reproduce the effects of PLAP and rPLAP described in this disclosure “AP” means alkaline phosphatase when there is no need for further specification.

The term “highly purified” means a preparation of PLAP prepared from human placenta or another tissue (in case of other APs) or produced by a recombinant method which contains less than 2% of contaminant proteins as determined by using a standard sodium dodecyl sulfate (SDS) gel electrophoresis protein separation method coupled with the commonly used Coomassie blue staining method and a densitometer for quantification of gel-bound stained proteins.

The term “therapeutically effective amount or dose” means a dose of rPLAP or PLAP that effectively, in a statistically significant manner, reduces chemotherapy induced loss of red blood cells, white blood cells, platelets,

In this disclosure, “chemotherapy” means any chemotherapeutic drug and their combinations, non-targeted or targeted, synthesized by a chemical method or produced by recombinant methods like antibodies targeted against specific antigens on the surface of cancer cells, that are used in the clinical practice to treat cancer and that cause loss of blood cells.

The term “BM” cells mean nucleated bone marrow cells mostly composed of HSCs and some other minor fractions including mesenchymal stem cells and colony forming cells.

### SUMMARY OF THE DISCLOSURE

The primary goal of this disclosure is to provide recombinant human placental alkaline phosphatase (rPLAP), as defined under “Definitions”, or highly purified human placenta-derived placental alkaline phosphatase (PLAP) for reducing the loss of red blood cells, white blood cells, platelets and their progenitors in chemotherapy treated cancer patients.

In one embodiment, rPLAP or PLAP is administered to a cancer patient to reduce chemotherapy induced loss of blood cells (cytopenia) and increase the survival of hematopoietic and mesenchymal stem cells as well as progenitors of blood cells thus allowing administration of chemotherapy either at higher doses and/or with greater frequency resulting in its enhanced efficacy.

In another embodiment, rPLAP or PLAP is administered to a cancer patient to enhance the survival of transplanted hematopoietic stem cells and mesenchymal stem cells thus promoting repopulation of the bone marrow with blood cell forming progenitors resulting in increased formation of red blood cells, white blood cells, and platelets that, in combination, contribute to increased life expectancy of the cancer patient.

In the work leading to this disclosure, cyclophosphamide (Cyp) was used as the model chemotherapeutic drug. It is a major component of numerous antitumor combinations composed of other chemotherapeutic drugs used in the treatment of many different cancers. Cyp is also used in myeloablative procedures prior to stem cell transplantation. Thus, Cyp can be regarded as a representative of all chemotherapeutic drugs that cause reductions in the number of red blood cells, white blood cells, and platelets as well as their progenitors.

### DETAILED DESCRIPTION

### 1. Placental Alkaline Phosphatase; the Active Component.

Humans express four dimeric AP enzymes (E.C.3.1.3.1); the placental (PLAP), the intestinal (IALP), the tissue nonspecific (TNAP), and the germ cell (GCAP) enzymes, each catalyzing the hydrolysis of phosphomonoesters accompanied by the release of inorganic phosphate and alcohol [Millan, J. L. Alkaline phosphatases: Structure, substrate specificity and functional relatedness to other members of a large superfamily of enzymes. Purinergic Signalling, 2, 335-341; 2006; Kozlenkov, A., Manes, T., Hoylaerts, M. F. and Millan, J. L. Function assignment to conserved residues in mammalian alkaline phosphatases. J. Biol. Chem. 277, 22992-22999, 2002]. PLAP and GCAP are closely related (— 95% homology). The sequence homology between human IALP and PLAP is about 86.5% [Henthorn, P. S., Raducha, M., Edwards, Y. H., Weiss, M. J., Slaughter, C. and Harris, H. Nucleotide and amino acid sequences of human intestinal alkaline phosphatase: Close homology to placental alkaline phosphatase. Proc. Natl. Acad. Sci. U.S.A. 84, 1234-1238, 1987], and somewhat less between bovine (and calf) IALP and PLAP [Weissig, H., Schildge, A., Hoylaerts, M. F., Iqbal, M. and Millan, J. L. Cloning and expression of the bovine intestinal alkaline phosphatase gene: Biochemical characterization of the recombinant enzyme. Biochem. J. 290, 503-508, 1993]. TNAP is expressed in the bone, liver and kidney and is about 50% or more identical with the other three human APs. Various APs are expressed from bacteria to humans with the main features of enzyme's properties being conserved [Millan, J. L. Alkaline phosphatases: Structure, substrate specificity and functional relatedness to other members of a large superfamily of enzymes. Purinergic Signalling, 2, 335-341, 2006] strongly suggesting that other human and non-human APs may reproduce, at least in part, the biological effects of PLAP described in this disclosure.

PLAP may be produced in a recombinant form (rPLAP). The rPLAP used in this disclosure the 27-amino acid long glycosylphosphatidylinositol (GPI) anchor is replaced with a shorter FLAG sequence to ensure efficient secretion of the proteins from cells they are produced in. This is a significant modification of PLAP which provides excellent examples that the structure of PLAP may be significantly modified without losing its ability to stimulate cell proliferation in vitro and most probably in vivo as well. Accordingly, it is expected that some other recombinant forms of PLAP and other APs can be generated that will reproduce, at least in part, the effects of PLAP, as described in the Examples in details. In this disclosure, highly purified native PLAP may be referred to as “PLAP” to distinguish it from the recombinant derivative of PLAP (rPLAP).

Site-specific mutations may be introduced into PLAP thus creating new derivatives that do not alter the catalytic activity but cause changes in its membrane binding [Lowe, M. E. Site-specific mutations in the COOH-terminus of placental alkaline phosphatase: a single amino acid change converts a phosphatidylinositol-glycan-anchored protein to a secreted protein. J. Cell Biol. 116, 799-807, 1992]. Such altered PLAPs are produced using recombinant methods thus they qualify as rPLAP.

This disclosure can also use an alkaline phosphatase (AP) which is a hybrid derivative of two APs. For example, in such a hybrid one critical segment may originate from PLAP accounting for the heat stability and stability in the circulation, and another segment may originate from a different AP accounting for a higher catalytic phosphatase activity or another useful property. As an example, envisioned in the disclosure, such hybrid may contain up to roughly 50% of the sequence of PLAP providing stability, and up to roughly 50% of the sequence of another human AP providing the catalytic alkaline phosphatase activity. The rationale for using such hybrid is that of the APs, PLAP has by far the greatest stability in the circulation, while other APs may have greater catalytic activities towards some specific substrates.

Recombinant methods for obtaining appropriate preparations of PLAP and other APs are feasible. For example, using the cDNA of PLAP, recombinant protein may be produced by one of the many known methods for recombinant protein expression. PLAP has been cloned and expressed in different cell types [Kozlenkov, A., Manes, T., Hoylaerts, M. F. and Millan, J. L. Function assignment to conserved residues in mammalian alkaline phosphatases. J. Biol. Chem. 277, 22992-22999, 2002; Henthorn, P., Zervos, P., Raducha, M., Harris, H. and Kadesh, T. Expression of a human placental alkaline phosphatase gene in transfected cells: Use as a reporter for studies of gene expression. Proc. Natl. Acad. Sci. USA 85, 6342-6346, 1988; Chen, Y. H., Chang, T. C. and Chang, G. G. Functional expression, purification, and characterization of the extra stable human placental alkaline phosphatase in the Pichia pastoris system. Protein Expression & Purification 36, 90-99, 2004; Becerra-Artega, A., Mason, H. S. and Shuler, M. I. Production, secretion, and stability of human secreted alkaline phosphatase in tobacco NT1 cell suspension cultures. Biotechnol. Prog. 22, 1643-1649, 2006]. Slightly modified forms of PLAP may be expressed in and obtained from other cell lines of human or animal origin, cow's milk, goat's milk, chicken egg, bacteria, and certain plant (for example, barley, rice, corn, wheat, tobacco) seeds or leaves.

If rPLAP is derived from plants that are used for human consumption without restriction, a protein extract from such source may be used, after careful testing, for oral consumption without further purification of the protein. Any of the available suitable extraction methods known in the food industry can be used to produce such protein extracts from plants.

A preparation of human PLAP may be obtained by extraction from placental tissue. Human placenta synthesizes the enzyme during pregnancy, so that toward the end of the third term the level of PLAP in the placenta tissue and the maternal and fetal blood becomes high compared to other APs. Therefore, a preparation of PLAP may be obtained by butanol extraction of homogenized placenta. Other methods of extraction from placental tissue are also suitable. Tissue specific APs other than PLAP may also be extracted and purified from blood, liver, and other tissues of human or animal origins.

### 2. The Method of Treatment.

### 2/A. The Subjects Treated.

The subjects to be treated with rPLAP or PLAP are male or female cancer patients of any age diagnosed with cancer and scheduled to receive chemotherapy that, as serious side effect, causes reductions in red blood cells, white blood cells, and platelets. The primary purpose of PLAP treatment is to reduce chemotherapy induced loss of these blood cells allowing the use of larger doses and more frequent applications of the chemotherapy resulting in enhanced life expectancy of cancer patients.

The half-life time of purified PLAP in human circulation is about 7 days [Clubb, J. S., Neale, F. C. and Posen, S. (1965) The behavior of infused human placental alkaline phosphatase in human subjects. J. Lab. & Clin. Med. 66, 493-507, 1965] and that of rPLAP used in the disclosure is about 10 days (unpublished observation) which is much longer than the half-life time of other APs that can be between few hours and one day. This beneficial feature which allows less frequent applications (e.g., three times a week, twice a week, once a week and once every second week) makes PLAP the preferred AP to use for reducing chemotherapy induced loss of blood cells.

Numerous Cyp containing chemotherapy compositions whose effects on the loss of blood cells can be reduced by PLAP are in the clinical practice. PLAP and rPLAP may also be used to reduce the effects of other prominent anticancer drugs on the loss of blood cells the list (that do not represent the full list) including platinum drugs (for example, cisplatin, carboplatin, oxaliplatin), doxorubicin, epirubicin, altretamine, azacitidine, bleomycin, busulfan, capecitabine, carmustine, chlorambucil, cladribine, clofarabine, cytarabine, dacarbazine, dactinomycin, daunorubicin, decitabine, etoposide, fluoroacyl, fludarabine, hydroxyurea, idarubicin, ifosfamide, irinotecan, mechlorethamine, methotrexate, melphalan, mitomycin, mercaptopurin, mitoxantrone, nelarabine, paclitaxel, docetaxel, lomustin, gemcitabine, gemtuzumab ozogamicin, mercaptopurine, pentostatin, procarbazine, raltitrexed, streptozocin (also named streptozotocin), teniposide, thiotepa, topotecan, or Valrubicin.

Most, if not all, chemotherapeutic agents are used in cycles allowing enough time for the recovery of bone marrow function. However, since one of the goals is to reduce the length between the cycles of chemotherapy treatments, PLAP or rPLAP is preferentially used continuously, via injection in a physiologically acceptable carrier, three times a week, twice a week, once a week, or biweekly regardless the length of time between two cycles of therapy.

In the practice of this disclosure, PLAP may also be used to enhance successful transplantation of hematopoietic stem cells (HSCs). In this procedure, the cancer cells and most normal cells residing in the bone marrow of the recipient are first destroyed (myeloablation) by high dose alkylating drug (such as Cyp) or an alkylating drug containing composition with or without using a radiation procedure prior to the transplantation event. The purpose of such extreme treatment is to attempt to eliminate all cancerous cells in the bone marrow (leukemia or other blood cancer cells, or metastasized tumor cells derived from solid tumors). This procedure is followed within a short period of time first by the injection of PLAP or rPLAP (in a range of between 0.005 to 2.5 g per m², or between 0.05 to 1 g per m², or between 0.1 to 1 g per m² of the calculated body surface of the cancer patient). and then by the transplantation of bone marrow HSCs, with or without MSC support, with the purpose of restoring blood cell formation. as rapidly as possible. The results of PLAP enabled faster recovery of blood cells are reduced occurrence of anemia as well as reduced infections and thrombocytopenia. These effects, demonstrated by data in the “Examples”, clearly indicate that in mice treated with high-dose chemotherapy for myeloablation, administration of PLAP significantly promoted formation of blood cells obviously requiring previous expansion of transplanted stem cells.

Adult HSCs used for re-populating the empty bone cavity may be obtained directly from the bone marrow (for example, from posterior iliac crests), or from peripheral blood. In the latter case, the donor (the patient himself/herself or a close relative) may be pretreated with G-CSF (granulocyte-colony stimulating factor) and/or GM-CSF (granulocyte/macrophage-colony stimulating factor) to mobilize bone marrow stem cells and enhance the yield of peripheral blood stem cells. However other methods may also be applied for the mobilization of stem cells, for example using compositions that include Cyp. The stem cell population may be enriched by various methods, for example by using magnetic-activated cell sorting to remove monocytes or T-lymphocytes or Ficoll-Hypaque density gradient centrifugation. Prior to transplantation, the stem cells are usually stored in a 5-20% dimethylsulfoxide-containing medium such as Iscove's modified Dulbecco's medium in the vapor phase of liquid nitrogen. Any standardized procedure for the isolation, enrichment and storage of stem/progenitor cells that are well known in the art may be used.

In most cases, transplantation of the stem cells will likely to be a single event due to the limited supply of cells and for safety reasons. After the subject receives bone marrow-derived stem cell transplantation, PLAP may be administered three times a week, twice a week, once a week, or once every second week for up to about 40 days.

In some treatments, however, PLAP may not be applied before or during the preparatory high dose chemotherapy treatment because it may interfere with the process.

### 3/B. Treatment Via a Systemic Route.

In this disclosure, rPLAP or PLAP is administered to the mammal or human by injection. Any suitable injection method, for example intravenous, subcutaneous, intraarterial, intramuscular, intraperitoneal, intraportal, or intradermal may be used. rPLAP or PLAP may also be administered via infusion or using an implanted device for controlled delivery.

For injection delivery, rPLAP or PLAP may be dispersed in any physiologically acceptable carrier that does not cause an undesirable physiological effect and ensures proper distribution of PLAP into the desired area. Examples of suitable carriers include physiological saline and phosphate-buffered saline. rPLAP or PLAP may also be attached to nanoparticles and then dispersed in a suitable carrier. The injectable solution may be prepared by dissolving or dispersing a suitable preparation of rPLAP or PLAP in the carrier using conventional methods. As an example, a suitable composition for the practice in the method comprises rPLAP or PLAP in a 0.9% physiological salt solution to yield a total protein concentration of 1 mg/ml. Another suitable composition contains rPLAP or PLAP in a 0.9% physiological salt solution to yield a total protein concentration of 10 mg/ml. A third composition contains alkaline phosphatase in a 0.9% physiological salt solution to yield a total protein concentration of 50 mg/ml. As an alternative method, rPLAP or PLAP may be enclosed in liposomes, such as immunoliposomes, or attached to other delivery systems or formulations, such as nanoparticles. These formulations are well known in the art.

A suitable dosage for systemic administration may be calculated in grams of the active agent(s) per square meter of body surface area for the subject. In one embodiment, the therapeutically effective amount is between 0.005 to 2.5 g of rPLAP or PLAP per m² body surface of the mammal. In another embodiment, the therapeutically effective amount of rPLAP or PLAP is between 0.05 to 1 g per m² body surface of the mammal. In yet another embodiment, the therapeutically effective amount of rPLAP or PLAP is between 0.1 to 1 g per m² body surface of the mammal. Administration of rPLAP or PLAP is performed two hours or less prior to delivering the transplant.

As for the timing of delivery, the therapeutically effective amount of rPLAP or PLAP may be administered three times a week, twice a week, once per week, or biweekly.

Another factor to consider when determining the effective amount of PLAPs is that it will be used as part of a more complex regimen involving other treatments with different characteristics and toxicity profiles. As the general rule, if the toxicity of the chemotherapy is greater than that caused by average treatment, then PLAPs may have to be used three times or twice a week instead of once a week or biweekly.

PLAPs are preferably administered prior to a major meal. Other medication(s) or substance(s) used to treat a disease may be administered as prescribed without affecting the schedule of rPLAP or PLAP administration.

In embodiments of the disclosure, PLAPs may be used together with other agents or enhancers that positively influence(s) the survival and proliferation of stem cells and progenitors in the bone marrow. Examples for such agents or enhancers include cytokines and growth factors such as G-CSF, GM-CSF, erythropoietin, interleukin-3, interleukin-11, IGF-1, insulin, growth hormone, platelet-derived growth factor, fibroblasts growth factor, placental growth factor, epidermal growth factor, vascular endothelial growth factor, transforming growth factors, transferrin, al-antitrypsin, testosterone, single amino acids like leucine or multi-component amino acid mixtures. In addition, PLAP may also be administered together with nutraceuticals, such as freeze-dried blueberry extract, green tea extract, black tea extract, carnosine, catechin, choline, serine, ethanolamine, inositol, or the activated form of vitamin D3. The above enhancers may be applied together or separately from PLAPs to enhance survival and proliferation of endogenous and transplanted bone marrow stem cells.

### EXAMPLES

### Example 1. Purification and Spectrophotometric Assay of PLAP

Human PLAP (Type XXIV, 1020 units of total activity) in a partially purified form was obtained commercially from Sigma-Aldrich. A butanol extraction of placental tissue performed by Sigma-Aldrich to obtain the partially purified material was followed by chromatography steps as described in [Chang, T.-C., Huang, S.-M, Huang, T.-M. and Chang, G.-G. Human placental alkaline phosphatase: An improved purification procedure and kinetic studies. Eur. J. Biochem. 209, 241-247, 1992] to obtain an essentially pure preparation of PLAP as determined by sodium dodecyl sulfate-polyacrylamide gel electrophoresis (SDS-PAGE). The enzyme activity of PLAP was assayed using a spectroscopic method to monitor the hydrolysis of 4-nitrophenylphosphate (as an increase in absorbance at 410 nm) at room temperature (22° C.) as described in [Chang, G.-G., Shiao, M.-S., Lee, K.-R. and Wu, J.-J., Modification of human placental alkaline phosphatase by periodate-oxidized 1,N6-ethenoadenosine monophosphate. Biochem. J., 272, 683-690, 1990]. Activity analysis of 5-10-μg purified enzyme was performed in 1 mL incubation volume containing 50 mM Na₂CO₃/NaHCO₃, 10 mM MgCl₂, 10 mM 4-nitrophenylphosphate at pH 9.8. The extinction coefficient of 4-nitrophenol was taken as 1.62×10⁴ M⁻¹ cm⁻¹. An enzyme activity of 1 U (unit) is defined as 1 μmole substrate hydrolyzed per 1 min at 22° C. at pH 9.8. The pure PLAP was further identified by sequence analysis performed by the Mayo Clinic Protein Core Facility (Rochester, MN, US). The specific activity of purified PLAP was 685 Units per mg protein.

### Example 2. Production of Recombinant PLAP

Recombinant PLAP (rPLAP) with full catalytic activity was produced by a method described by others [Kozlenkov, A., Manes, T., Hoylaerts, M. F. and Millan, J. L. Function assignment to conserved residues in mammalian alkaline phosphatases. J. Biol. Chem. 277, 22992-22999, 2002]. In rPLAP the 27-amino acid long glycosylphosphatidylinositol (GPI) anchor (present in purified PLAP) is replaced with an 8-amino acid long FLAG sequence at the carboxyl terminal to ensure efficient secretion of the protein.

### Example 3. Development of T47 Human Breast Cancer Tumors

T47D estrogen receptor positive human breast cancer cells were from American Tissue Culture Collection (10801 University Boulevard, Manassas, Virginia, 20110-2209, United States). The tumor pieces were maintained in mouse tail. In the following in vivo experiments, tumor pieces containing about 1.5×10⁶ tumor cells were implanted subcutaneously into the intrascapular region to develop the tumors.

### Experiments Performed In Vitro

### Example 4. Combined Effects of PLAP, ATP, and Cyp on DNA Synthesis in MSCs

Inflammation of the bone marrow and resulting cell death caused by chemotherapy results in the release of ATP from damaged and/or dying cells. ATP inhibits proliferation of MSCs via purinergic receptors. Next it was tested if PLAP could modify the inhibitory effects of ATP on the proliferation of MSCs.

For the experiment, bone marrow aspirates were taken from normal human adult donors after informed consent. For the preparation of bone marrow MSCs, essentially a widely used technique was used as described earlier by others [Pittenger, M. F., Mackay, A. M., Beck, S. C., et al. (1999) Multilineage potential of adult human mesenchymal stem cells. Science 284, 143-147]. Briefly, nucleated cells were isolated with a pre-prepared commercial density gradient (Lymphoprep, Nycomed, Pharma, Oslo, Norway) and re-suspended in Dulbecco's modified Eagle's medium (DMEM) (GIBCO, Grand Island, NY) supplemented with 10% fetal calf serum (FCS), 50 U/ml of penicillin, and 50 μg/ml of streptomycin (GIBCO). All nucleated cells were plated in 25-cm² flasks (BD Falcon, Bedford, MA) at 37° C. in humidified atmosphere containing 5% CO₂. After 24 hours, non-adherent cells were removed and cryopreserved in liquid nitrogen until use. The remaining adherent cells were thoroughly washed with Hanks balanced salt solution (HBSS) (GIBCO). Fresh complete culture medium was added and replaced every 3 or 4 days (twice a week). When cells grew to about 80% confluence, they were suspended and harvested by incubating with a ready-made solution containing 0.25% trypsin and 1 mM EDTA (Sigma-Aldrich, St. Louis, MO) for 5 minutes at 37° C.; this cell suspension is designated as passage 1. These cells were further expanded with 1:3-1:5 splitting in 175-cm² flasks (BD Falcon). For the determination of DNA synthesis, cells were divided into 12-well plates in the complete medium and let the cells grow to about 60% confluence. After changing the spent medium for fresh complete medium (i.e., containing 10% fetal calf serum), cells were treated for 20 hours with 20-100 μM ATP and/or 200 nM PLAP as well as 50-200 μM Cyp in the absence or presence of 200 nM PLAP. For labeling newly formed DNA, cells received 1,130,000 d.p.m. [³H] thymidine for the last 4 hours of the treatment period. DNA synthesis was determined as described earlier [She, Q.-B., Mukherjee, J. J., Huang, J.-S., Crilly, K. S. and Kiss, Z. (2000) Growth factor-like effects of placental alkaline phosphatase in human fetus and mouse embryo fibroblasts. FEBS Lett. 469, 163-167]. The data is expressed as d.p.m. [³H] thymidine incorporated into DNA/well (n=3).

The data, shown in TABLE 1, confirms earlier findings [Coppi, E., Pugliese, A. M., Urbani, S. et al. (2007) ATP modulates cell proliferation and elicits two different electrophysiological responses in human mesenchymal stem cells. Stem Cells. 25, 1840-1849, 2007] that ATP inhibits proliferation (as indicated by DNA synthesis) of MSCs. Importantly, in these cells PLAP prevented the inhibitory effect of ATP on DNA synthesis. In addition, the data shows that Cyp also inhibited proliferation of MSCs (perhaps due to reduced cell viability or increased cell death) which was significantly reversed by PLAP.

TABLE 1
Combined effects of PLAP, ATP, and
Cyp on DNA synthesis in MSCs.

| | d.p.m. [³H] thymidine incorporated into DNA/well | |
|---|---|---|
| Treatments | No PLAP | +PLAP, 200 nM |
| No ATP | 79,033 ± 2,855 | 89,865 ± 2,021* |
| ATP, 20 μM | 70,738 ± 1,367 | 90,708 ± 2,518* |
| ATP, 50 μM | 55,808 ± 1,480 | 84,740 ± 3,231** |
| ATP, 100 μM | 38,601 ± 1,940 | 80,916 ± 2,252** |
| Cyp, 50 μM | 51,684 ± 2.772 | 81,887 ± 3,173** |
| Cyp, 200 μM | 32,536 ± 3,280 | 74,798 ± 2,408** |

*Significantly (P < 0.01) greater than “No ATP or “ATP, 20 μM”.
**Significantly (P < 0.001) greater than corresponding values with “no PLAP”.

### Example 5. Effects of PLAP on the Proliferation of Mouse Bone Marrow-Derived Hematopoietic Stem Cells (HSCs) in Vitro

Bone marrow was harvested by flushing bones from female C57/BL/6 mice in Fischer medium (Thermo Fisher Scientific; catalogue no. 21475025) containing 20% horse serum (Hyclone, Logan, UT), 100 Unit/ml penicillin, 100 kg/ml streptomycin (Gibco, BRL), 0.015 fungizone (Gibco, BRL), and 0.1 μM hydrocortisone sodium succinate (SynoPharm; Hamburg, Germany). Pooled stem cells were seeded into 25 cm 3 flasks (10 ml cell suspension) and after 3 days of incubation at 33° C. in 5% CO₂ in air in the absence of PLAP, they were plated into 12-well plates and were incubated in the absence (n=8) or presence of 50 μg/ml PLAP (n=8) for additional 3 days. Such treatment did not significantly change the number of hematopoietic stem cells as determined by a standard method using a hemocytometer.

In a follow up experiment with hematopoietic stem cells, the same incubation medium in 12-well plates contained either no other addition (n=8) or contained 200 μM Cyp without (n=8) or with 50 μg/ml PLAP (n=8) for 3 days. In absence and presence of PLAP, Cyp decreased the number of cells by 52% and 10%, respectively.

These observations suggest that while PLAP may not directly enhance proliferation of hematopoietic stem cells, it is likely to increase survival of both endogenous and transplanted hematopoietic bone marrow stem cells during chemotherapy treatment. The bone marrow environment after chemotherapy is characterized by low cell density and, consequently, by low levels of growth and survival factors. All this considered, PLAP-promoted survival of hematopoietic stem cells likely to contribute to earlier restoration of bone marrow cellularity and blood formation.

### Experiments Performed In Vivo

### Example 6. Effects of Subcutaneously Administered PLAP on the Blood Profile in Cyp-Treated T47D Human Breast Tumor Bearing Mice

Life threatening bone marrow toxicity is the primary reason why chemotherapy is delivered in cycles, with weeks between them, allowing recovery of bone marrow function between cycles. Because of that, chemotherapy cannot be applied at doses or frequencies that would be required for optimum anticancer effects. Thus, a bone marrow cell protective agent promoting the survival of progenitors and formation of red blood cells, white blood cells, and platelets would allow the use of higher doses and/or more frequent applications of chemotherapy; in short, it would increase both the efficacy of chemotherapy and life expectancy of cancer patients. The following two experiments served to test whether PLAP had positive effects on blood cells in Cyp treated tumor bearing mice.

It should be noted that chemotherapy is known to create a highly proteolytic microenvironment in the bone marrow which goes along with a decrease of endogenous protease inhibitors [Maurer, A., Klein, G. and Staudt, N. D. Assessment of proteolytic activities in the bone marrow microenvironment. Methods Mol. Biol. 2017, 149-163, 2019]. This process may reduce the concentration of PLAP in the bone marrow to such a low level that it may not be able to exhibit any protective effects such as described for MSCs and HSCs in TABLE 1 and subsequent text. For this reason, the outcome of rPLAP effects on the formation of blood cells was completely unpredictable.

In the two experiments performed, first-generation hybrid BDF1 (C57BL/6 female×DBA/2 male) 10 weeks old adult female mice bearing T47D tumors (2.6-3.3 cm 3 volume) were used.

### Experiment 1. Effects of PLAP on the Blood Cell Counts in Cyp-Treated T47D Tumor Bearing Mice, the Last Blood Sample Taken on Day 9

In the first group, mice received no treatment. In the second and third groups, mice were administered 120 mg/kg of cyclophosphamide (Cyp) intraperitoneally once a day for 6 consecutive days administered between 10-10.30 AM. In a third group, mice were also administered 7.5 mg/kg of PLAP subcutaneously on days 0, 3 and 6 between 9-9.30 AM. In each group, mice were kept on standard diet (˜15% fat, 65% carbohydrate, and 20% proteins) purchased from Charles River VRFI. Both PLAP (for the 3rd group) and Cyp were dissolved in physiological (0.9%) saline and injected in 50 μl volume. On the days indicated in TABLE 2, blood samples were collected from the ocular venous plexus of ether-anesthetized mice by retro-orbital venipuncture in heparin treated tubes followed by counting the blood cells with a Sysmex F-800 hematology analyzer (Toa Medical Electronics Co. LTD, Kobe, Japan). The data shown is the average of 6 independent determinations ±S.D.

Data in TABLE 2 shows that Cyp treatment greatly reduced the counts of red blood cells (RBCs), white blood cells (WBCs), and platelets, and in each case PLAP significantly reduced the loss of blood cells on both day 6 and day 9. These results mean that PLAP can (1) help maintain oxygen supply for tissues at a higher level than afforded by Cyp treatment alone, (2) significantly reduce infections due to its ability to keep WBCs in a healthier range, and (3) help formation of blood clots and thereby reduce bleeding by preventing large decreases in platelet numbers (thrombocytopenia).

TABLE 2
PLAP reduced loss of blood cells in
Cyp treated T47D tumor bearing mice.

| | | | |
|---|---|---|---|
| | Untreated | Cyp | Cyp + PLAP |
| Days | First group | second group | third group |

RBCs, ×10⁻⁶/μl

| | | | |
|---|---|---|---|
| 0 | 9.27 ± 1.01 | 9.51 ± 1.09 | 9.10 ± 0.82 |
| 3 | — | 7.28 ± 1.29 | 8.20 ± 1.00 |
| 6 | — | 4.94 ± 0.63* | 6.79 ± 0.52** |
| 9 | — | 6.26 ± 0.71* | 9.01 ± 0.78** |

WBCs, ×10⁻³/μl

| | | | |
|---|---|---|---|
| 0 | 8.29 ± 0.35 | 7.92 ± 0.72 | 8.05 ± 0.31 |
| 3 | — | 4.22 ± 0.85* | 6.37 ± 0.97 |
| 6 | — | 2.73 ± 0.53* | 6.15 ± 0.65** |
| 9 | — | 5.72 ± 0.49* | 7.94 ± 0.70** |

Platelets, ×10⁻³/μl

| | | | |
|---|---|---|---|
| 0 | 1,155 ± 118 | 1,114 ± 111 | 1,152 ± 101 |
| 3 | — | 704 ± 98* | 888 ± 112 |
| 6 | — | 404 ± 139* | 867 ± 144** |
| 9 | — | 687 ± 114* | 1,075 ± 107** |

*Significantly (P < 0.01) smaller than day 0.
**Significantly (P < 0.01) different from Cyp alone.

### Experiment 2. Effects of PLAP on the Blood Cell Counts in Cyp-Treated T47D Tumor Bearing Mice, the Last Blood Sample Taken on Day 10

The second experiment was performed with T47D tumor bearing mice similarly to the one described to TABLE 2, except that one group of Cyp (120 mg/kg) treated mice were co-treated treated with PLAP (7.5 mg/kg) on days 0, 2, 4, and 6. Blood was taken for analysis on days 0, 6, and 10, and blood cells were determined with a Sysmex F-800 hematology analyzer (Toa Medical Electronics Co. LTD, Kobe, Japan). The data shown is the average of 6 independent determinations±S.D.

Data in TABLE 3 shows that Cyp treatment again greatly reduced the counts of all three major blood cell types, and in each case PLAP prevented Cyp induced large drops in red blood cells (RBCs), white blood cells (WBCs), and platelets as measured on days 6 and 10.

These results confirm that PLAP can be used to compensate for chemotherapy induced large reductions in the counts of critically important blood cells, thereby reducing the risk of infections and bleeding as well as provide better oxygen supply for all tissues with the result of the treated cancer patients feeling less tired and most probably living longer.

TABLE 3
PLAP prevents large decreases in white blood cells and platelets
in the blood of Cyp treated T47D tumor bearing mice.

| | | | |
|---|---|---|---|
| Days | Untreated | Cyp | Cyp + PLAP |

RBCs, ×10⁻⁶/μl

| | | | |
|---|---|---|---|
| 0 | 9.36 ± 0.56 | 9.51 ± 0.70 | 9.60 ± 0.87 |
| 6 | — | 4.83 ± 0.62* | 6.51 ± 0.53 |
| 10 | — | 6.88 ± 0.61* | 8.67 ± 0.57** |

WBCs, ×10⁻³/μl

| | | | |
|---|---|---|---|
| 0 | 8.55 ± 0.36 | 8.86 ± 0.61 | 8.36 ± 0.63 |
| 6 | — | 2.79 ± 0.47* | 6.91 ± 0.66** |
| 10 | — | 6.15 ± 0.45* | 7.97 ± 0.56** |

Platelets, ×10⁻³/μl

| | | | |
|---|---|---|---|
| 0 | 1,223 ± 93 | 1,185 ± 101 | 1,219 ± 95 |
| 6 | — | 372 ± 57* | 836 ± 118** |
| 10 | — | 746 ± 92* | 1,158 ± 102** |

*Significantly (P < 0.01) smaller than day 0.
**Significantly (P < 0.01) greater than “Cyp” alone.

### Example 7. Effects of PLAP on Nucleated Bone Marrow Cells (BM) and Granulocyte-Macrophage Colony-Forming Units (CFU-GM) in Cyp Treated T47D Tumor Bearing Mice without Stem Cell Support

Again, first-generation hybrid BDF1 (C57BL/6 female×DBA/2 male) 10 weeks old adult female mice bearing T47D tumors (2.6-3.3 cm³) were used. In the first group, mice received no treatment. In the second and third groups, mice were administered 120 mg/kg of Cyp intraperitoneally once a day for 6 consecutive days. In a third group, mice were also administered 7.5 mg/kg of PALP subcutaneously on days 0, 2, 4 and 6. On the days indicated in TABLE 4, the animals were killed, then the bone marrow was flushed from the femurs with Iscove's modified Dulbecco's medium (Gibco BRL, Gaithersburg, MD, USA). This was followed by standard erythrocyte lysis and then by counting the number of nucleated bone marrow (BM) cells per femur using a hemocytometer.

To measure the formation of the granulocyte-macrophage colony-forming units (CFU-GM), a semisolid colony-forming cell assay was used. Equal numbers of nucleated bone marrow cells (isolated on days as indicated above) were plated in 35-mm Petri dishes (Costar, Cambridge, MA, USA) in Iscove's modified Dulbecco's medium supplemented with 1% methylcellulose, 30% horse serum (Gibco), 10% WEHI-3B conditioned medium as a source of growth factors, 4 mM L-glutamine, 0.25 mM α-thioglycerol (Gibco), 1% deionized bovine serum albumin (Sigma), and antibiotics (Gibco). Cells were cultured at 37° C. in 5% CO₂/95% air atmosphere. CFU-GM was counted on day 9. Three parallel plates were used for each 3 animals (9 plates for one number). Colonies containing at least 50 cells were counted which is a generally accepted value in the literature. In TABLE 4, “CFU-GM per femur” is a calculated value derived from multiplying the number of nucleated cells in the femur with the number of CFU-GM; this number gives the number of CFU-GM in one femur. Each value is expressed as the mean±standard deviation of 9 plates (derived from 3 animals).

As shown in TABLE 4, in the T47D tumor model Cyp caused a large reduction in nucleated bone marrow cells (BM cells) by day 6 which has not significantly improved by day 9. Similarly, formation of the granulocyte-macrophage colony-forming units (CFU-GM) per femur, although improved between day 6 and 9, remained well below the normal value in the Cyp treated mice. In contrast, by day 9, co-treatment with PLAP resulted in nearly complete recovery of BM cells and even an overshoot of the formation of CFU-GM per femur.

TABLE 4
PLAP promotes recovery of bone marrow function
in Cyp treated T47D tumor bearing mice.

| | | | |
|---|---|---|---|
| Days | Untreated | Cyp | Cyp + PLAP |

Nucleated BM cells/femur (×10⁶)

| | | | |
|---|---|---|---|
| 0 | 15.10 ± 1.10 | 14.11 ± 1.50 | 13.88 ± 0.82 |
| 3 | — | 9.32 ± 2.32* | 10.76 ± 1.10 |
| 6 | — | 3.10 ± 2.26* | 5.45 ± 2.03* |
| 9 | — | 4.90 ± 2.17* | 12.40 ± 2.37** |

CFU-GM/10⁵ nucleated BM cells

| | | | |
|---|---|---|---|
| 0 | 77.5 ± 6.1 | 78.1 ± 6.1 | 76.1 ± 6.4 |
| 3 | — | 34.5 ± 7.2* | 44.5 ± 5.7 |
| 6 | — | 25.8 ± 4.4* | 33.6 ± 4.9** |
| 9 | — | 98.5 ± 8.0 | 118.5 ± 7.4** |

CFU-GM per femur

| | | | |
|---|---|---|---|
| 0 | 1,170 | 1,101 | 1,056 |
| 3 | — | 321 | 478 |
| 6 | — | 80 | 183 |
| 9 | — | 482 | 1,469 |

*Significantly (P < 0.01) smaller than “day 0”.
**Significantly (P < 0.05-0.01) greater than “Cyp”

### Example 8. rPLAP Enhances Recovery of Blood Cells in Cyp+HSC Treated Tumor Bearing Mice

In some cases of blood cancers (leukemias, lymphomas) or when solid cancers (like prostate cancer) are metastasized to the bone marrow, a myeloablative procedure followed by transplantation of HSCs to repopulate the bone marrow is used. The extent of repopulation of bone marrow depends on how many viable niche forming cells (like MSCs, adipocytes, etc.), cancer cells, ATP, and growth factors remain after the myeloablative procedure. It is an important goal that restoration of production of red blood cells, white blood cells, and platelets occurs within the shortest time possible to avoid shortage of oxygen supply, infections, and internal bleeding. The next experiment served to examine if rPLAP was suitable for that purpose.

Myeloablative procedures cause multitude of effects on the remaining structural elements of bone marrow. In addition, chemotherapy creates a highly proteolytic microenvironment in the bone marrow which goes along with a decrease of endogenous protease inhibitors [Maurer, A., Klein, G. and Staudt, N. D. Assessment of proteolytic activities in the bone marrow microenvironment. Methods Mol. Biol. 2017, 149-163, 2019]. These two processes may reduce the concentration of PLAP in the bone marrow to such a low level that it may not be able to exhibit any protective effect on blood cells and their progenitors. For these reasons, the outcome of rPLAP effects on the formation of blood cells was completely unpredictable.

In this experiment, 14-week-old T 47 tumor bearing C57BL/6 mice weighing 28-30 grams were used. HSCs were prepared as described in [Spangrude, G. J., Heimfeld, S, and Weissman, I. L. Purification and Characterization of Mouse Hematopoietic Stem Cells. Science, 241, 4861-4865, 1988].

In the first group, mice received no treatment. In the second and third groups, mice were administered high dose (160 mg/kg) of Cyp intraperitoneally (i.p.) once a day for 3 consecutive days for the purpose of myeloablation. Then, on the 4th day, mice in the second and third groups received HSC transplantation (10⁶ cells per mouse). In the third group, on day 4 and 2 hours prior to stem cell transplantation mice were also administered 2.0 mg/kg body weight of rPLAP. rPLAP treatment was repeated on day 6 exactly 48 hours after the first treatment. rPLAP was dissolved in physiological (0.9%) saline and injected subcutaneously in 50 μl volume. Each group included 3 animals. Blood samples were collected on (i) day 0, 2 hours prior to Cyp treatment, (ii) day 4, 4 hours prior to stem cell transplantation, and (iii) day 8. Complete cell counts (RBCs, red blood cells; WBCs, white blood cells; platelets) were determined with a Sysmex F-800 hematology analyzer (Toa Medical Electronics Co. LTD, Kobe, Japan). All data shown in TABLE 4 is the average of 3 independent determinations±S.D.

Increase of Cyp dose from 120 mg/kg to 160 mg/kg resulted, by day 4, in even greater reductions in blood cell counts. Addition of rPLAP prior to stem cell transplantation facilitated recovery of normal blood profile in a statistically significant (P<0.05-0.01) manner (TABLE 5). All information considered in TABLE 2 through TABLE 5, it seems clear that in chemotherapy treated animals rPLAP and PLAP not only reduced the loss of red blood cells, white blood cells, and platelets but rPLAP, and most likely PLAP as well, can also enhance recovery of bone marrow's blood cell forming function from transplanted HSCs. The data imply that in chemotherapy treated cancer patients, both rPLAP and PLAP may be used to help the restoration of red blood cell, white blood cell, and platelet numbers.

TABLE 5
rPLAP enhances recovery of blood
cells in Cyp + HSCs treated mice.

| | | | |
|---|---|---|---|
| Day | Untreated | Cyp + HSCs | Cyp + HSCs + rPLAP |

RBCs, ×10⁻⁶/μl

| | | | |
|---|---|---|---|
| 0 | 9.17 ± 0.86 | 9.62 ± 0.62 | 9.50 ± 0.31 |
| 4 | — | 5.75 ± 0.46* | 6.05 ± 0.42 |
| 8 | — | 8.01 ± 0.29* | 9.25 ± 0.47** |

WBCs, ×10⁻³/μl

| | | | |
|---|---|---|---|
| 0 | 8.60 ± 0.41 | 8.72 ± 0.39 | 9.03 ± 0.67 |
| 4 | — | 1.79 ± 0.35* | 1.88 ± 0.42 |
| 8 | — | 5.54 ± 0.58* | 8.51 ± 0.77** |

Platelets, ×10⁻³/μl

| | | | |
|---|---|---|---|
| 0 | 1,128 ± 89 | 1,089 ± 92 | 1,184 ± 108 |
| 4 | — | 265 ± 46* | 282 ± 51 |
| 8 | — | 680 ± 72* | 1,103 ± 147** |

*Significantly (P < 0.01) smaller than “day 0”.
**Significantly (P < 0.01) greater than “Cyp + HSCs”.

1.-16. (canceled)

17. A method to manage chemotherapy care of a cancer patient comprising administering recombinant placental alkaline phosphatase or highly purified human placental alkaline phosphatase to the cancer patient to reduce chemotherapy-induced loss of red blood cells, white blood cells, or platelets, or combinations thereof.

18. The method of claim 17, wherein administration of recombinant placental alkaline phosphatase or placental alkaline phosphatase allows using chemotherapy at higher doses or with greater frequencies, or both using chemotherapy at higher doses and with greater frequencies.

19. The method of claim 17, wherein administering recombinant placental alkaline phosphatase or highly purified human placental alkaline phosphatase increases of chemotherapy efficacy.

20. The method of claim 17, wherein the placental alkaline phosphatase is isolated and purified from human placenta having a purity of 98% or greater.

21. The method of claim 17, wherein the recombinant placental alkaline phosphatase is produced by a mammalian cell, a bacterium, a yeast, or purified from cow's milk, goat's milk, chicken eggs, or plants.

22. The method of claim 21, wherein the sequence of the active recombinant placental alkaline phosphatase is altered compared to the full length placental alkaline phosphatase.

23. The method of claim 21, wherein the recombinant placental alkaline phosphatase comprises a hybrid protein containing up to about 50% of an amino acid sequence of placental alkaline phosphatase sufficiently long to enhance stability in the circulation, and up to about 50% of an amino acid sequence of another alkaline phosphatase sufficiently long to provide catalytic alkaline phosphatase activity.

24. The method of claim 17, wherein the recombinant placental alkaline phosphatase or placental alkaline phosphatase is administered to the cancer patient via injection in a physiologically acceptable carrier three times a week, twice a week, once a week, or biweekly independent of the length of time between two cycles of therapy.

25. The method of claim 24, wherein the physiologically acceptable carrier is 0.9% sodium chloride solution or a liposomal preparation or a nanoparticle preparation.

26. The method of claim 24, wherein the injection is intravenous, intra-arterial, intraperitoneal, intramuscular, intraportal, subcutaneous, or intradermal.

27. The method of claim 24, wherein the recombinant placental alkaline phosphatase or placental alkaline phosphatase is administered via infusion or by a controlled release device.

28. The method of claim 17, wherein the recombinant placental alkaline phosphatase or placental alkaline phosphatase is administered in therapeutically effective amounts in a range of between 0.005 to 2.5 g per m2, or between 0.05 to 1 g per m2, or between 0.1 to 1 g per m2 of the calculated patient's body surface.

29. The method of claim 17, wherein the recombinant placental alkaline phosphatase or placental alkaline phosphatase is administered via one a systemic route prior to, during, or after administration of any cancer medication or any other medication prescribed for an indication other than cancer.

30. The method of claim 17, wherein the chemotherapy administers cyclophosphamide or a cyclophosphamide containing drug composition.

31. The method of claim 17, wherein chemotherapy administers any anticancer drug or combinations with other anticancer drugs that cause the cancer patient's loss of red blood cells, white blood cells, platelets, or combinations thereof

32. A method to manage chemotherapy care of a cancer patient comprising administering recombinant placental alkaline phosphatase or placental alkaline phosphatase to the cancer patient before transplanting hematopoietic stem cells without or with mesenchymal stem cells to increase the viability of the stem cells to promote formation of red blood cells, white blood cells, platelets, or combinations thereof.
